(19) United States
(12) Patent Application Publication
(10) Pub. No.: US 2026/0233015 A1
(43) Pub. Date: Aug. 13, 2026

(54) SURGERY MODE ON-DEMAND

(71) Applicant: BIOTRONIK SE & CO. KG, Berlin (DE)

(72) Inventors: Hendrik DEUTSCHMANN, Berlin (DE); Jens MUELLER, Berlin (DE); Daniel KRUEGER, Bestensee (DE)

(73) Assignee: BIOTRONIK SE & CO. KG, Berlin (DE)

(21) Appl. No.: 19/150,834

(22) PCT Filed: Jan. 5, 2024
(86) PCT No.: PCT/EP2024/050210

§ 371 Date: Jul. 24, 2025

(30) Foreign Application Priority Data
Feb. 7, 2023 (EP) 23155335.5

(51) Int. Cl. A61N 1/372; A61N 1/08; A61N 1/39

(52) U.S. Cl. CPC A61N 1/37264; A61N 1/086; A61N 1/37247; A61N 1/3956

(57) ABSTRACT

An implant configured to provide a medical treatment to a patient and/or monitor a health state of the patient according to at least one standard operation parameter, when in a standard operation mode. The implant further includes means for receiving a transient mode request, wherein receiving the transient mode request causes the implant to switch, a single time, into a transient mode from a first time until a second time and to switch back to the standard operation mode thereafter. Further, the implant is configured to, when in the transient mode, provide the medical treatment to the patient and/or monitor the health state of the patient according to at least one transient parameter, wherein the at least one transient parameter is different from the at least one standard operation parameter.

### CROSS-REFERENCE TO RELATED APPLICATIONS

This application is the United States National Phase under 35 U.S.C. § 371 of PCT International Patent Application No. PCT/EP2024/050210, filed on Jan. 5, 2024, which claims the benefit of European Patent Application No. 2315535.5, filed on Feb. 7, 2023, the disclosures of which are hereby incorporated by reference herein in their entireties.

### TECHNICAL FIELD

The present invention relates to implants, apparatuses, systems, methods, and computer programs for transient mode operation of medical implants.

### BACKGROUND

Currently, implants may be re-programmed by an implant specialist during an appointment of the patient with the attending implant specialist, e.g., a health professional.

In one prominent example where this is necessary, surgery preparations need to be performed in-office, e.g., with the help of a cardiologist for patients with cardiac rhythm management (CRM) implants. In detail, if a surgery (both, cardiological and non-cardiological) is planned, e.g., for a patient with a CRM implant, at least two in-clinic cardiology consultations are needed, one prior to the surgery to assess therapy and change implant program to special therapy settings and one after the surgery to activate therapy again which was active before surgery. For example, the first consultation may comprise deactivating shock output for implantable cardioverter-defibrillators (ICDs) or safe ventricular (VVI) backup stimulation. This process is time demanding and inefficient for both, cardiologist and patient: Appointments need to be made, the medical staff must prepare for it, the patient needs to travel. All in all, this procedure takes days or weeks for an action that can be performed within minutes. Altogether, there is still a need to further improve workflows in terms of methods and involved apparatuses, implants, and respective systems.

This need is met at least in part by the aspects of the invention described herein.

The present disclosure is directed toward overcoming one or more of the above-mentioned problems, though not necessarily limited to embodiments that do.

### SUMMARY

According to an aspect of the invention, an implant is provided that is configured to provide a medical treatment to a patient and/or monitor a health state of the patient according to at least one standard operation parameter, when in a standard operation mode. The implant further comprises means for receiving a transient mode request, wherein receiving the transient mode request causes the implant to switch, a single time, into a transient mode from a first time until a second time and to switch back to the standard operation mode thereafter. Further, the implant may be configured to, when in the transient mode, provide the medical treatment to the patient and/or monitor the health state of the patient according to at least one transient parameter, wherein the at least one transient parameter is different from the at least one standard operation parameter.

Thereby, the implant may switch from its standard operation mode into the transient mode for a limited time. The transient mode may be tailored to any upcoming event that may require an adjustment of the operation of the implant. As the implant only switches into the transient mode for a limited time, this may save time consuming measures for, e.g., switching the implant back into its standard operation mode. Further, this renders a consultation for switching back the implant obsolete and may thus save time and reduce the workload of attending health professionals/implant specialists and the patient with the implant.

Herein, the transient mode may be scheduled, e.g., on demand and/or around a specific, non-recurring event like, e.g., a surgery during which the implant may be required to be set into a transient (surgery) mode, a flight during which the implant should be in a transient (flight) mode, etc. The first time may for example be before the start of the upcoming event for which the transient mode shall be scheduled, and the second time may for example be after the end of that event.

The transient mode and the standard operation mode may, e.g., be described by the same parameters which may have different values for the two modes. In one example, when there is only one parameter describing the standard operation mode, namely “on”, and only one parameter describing the transient mode, namely “off”, the transient mode may simply involve switching the respective function off for a predetermined time during the transient mode and then on again. In another example, when a plurality of parameters describes the two modes, one or more of the plurality of parameters may be different for the standard operation mode and the transient mode and, e.g., zero, one or more parameters may have the same value for the two modes. E.g., in case of the implant being a neurostimulation device, the standard operation mode may be described by two parameters determining the neurostimulation therapy delivered by the neurostimulation device, e.g., a frequency f_{SOM} at which neurostimulation pulses are delivered to the patient and an amplitude Asom of the delivered pulses. Switching the exemplary neurostimulation device of such example into a transient mode may, e.g., comprise reducing the frequency at which pulses are delivered in the transient mode f_{TM} for example to about 80% of the initial value or less, e.g. about 50% of the initial value (f_{TM}=f_{SOM}/2) or less and/or reducing the amplitude of the delivered pulses in the transient mode ATM to about 80% of the initial value or less, e.g. about 50% of the initial value (A_{TM}=A_{SOM}/2) or less. Any absolute or relative increase may occur. For non-number values any other suitable change (e.g., “on”/“off”) may be done. Generally, new parameters may be added in the transient mode that did not exist in the standard operation mode, e.g., in the example of a neurostimulation device the transient mode may be duty cycled, while the standard operation mode is not, and respective parameters may be introduced for the transient mode and vice versa. po For example, the parameters describing the transient mode may vary over time during the transient mode between the first and the second time. E.g., the transient mode may have different phases with different parameters. All these information may be comprised in such transient mode request.

Further, the implant may operate in the standard operation mode before the first time and after the second time. In the time in between, during the transient mode, the implant may, e.g., switch off completely, switch off part of its functions running in the standard operation mode, and/or change its operation according to the transient parameters. This may, e.g., be ensured by deactivating shock output for implantable ICDs in the transient mode or safe VVI backup stimulation.

Generally, the implant may, e.g., comprise a set of instructions describing a predetermined transient mode, e.g., by comprising the at least one transient parameter according to which the medical treatment is provided to the patient and/or the health state of the patient is monitored during the transient mode by the implant. The implant may further be configured to receive a transient mode request instructing the implant to schedule and/or to switch into such predetermined transient mode in a certain time window.

In an example, the implant may further be configured to discard the transient mode request such that, after switching back to the standard operation mode, the implant continues to remain in the standard operation mode without switching back to the transient mode again, unless another transient mode request is received.

This allows for example for a one-time adjustment for exceptional situations, e.g., scheduled surgeries, such that these may be performed without putting the patient and/or the attending health professional(s), e.g., a surgeon performing a surgery, at risk but allow for safe operation of the implant during the respective time window. As, however, typically an implant may, e.g., provide necessary assistance in keeping the patient in a certain physiological state, switching the implant back into its standard operation mode after such event, may be critical for the patient's health. Thus, this invention may significantly reduce the risk associated with waiting until a scheduled appointment with a responsible implant specialist/attending health professional, e.g., on the next day as in the meantime, the patient may suffer from not being provided with the required therapy provided by the implant.

For example, when an event is expected in a given time slot, the implant may switch from the standard operation mode into the transient mode, e.g., 2 h, 1 h, 30 min, 20 min, 10 min, 5 min, 0 min, or any other time before the start of that time slot and may switch back into the standard operation mode, e.g., 2 h, 1 h, 30 min, 20 min, 10 min, 5 min, 0 min, or any other time after the end of that time slot. Switching the implant into the transient mode even before the start of the time slot and switching it back into the standard operation mode after the end of the time slot may ensure that the implant is in the transient mode during the respective event even when the event associated with the time slot may start earlier and/or end later than initially expected when scheduling the transient mode.

In an example, the transient mode request may indicate the first time and the second time for one-time use.

Thereby, the implant may, upon receiving the transient mode request, be provided with all necessary information to be in the transient mode as required. This may be particularly relevant when, e.g., the first and/or second time are not identical with the start and end of the time slot associated with the upcoming event which the transient mode is being designed for.

In another example, the transient mode request may further comprise an instruction to use a predetermined mode of the implant as the transient mode.

The implant may accordingly comprise, e.g., at least one set of instructions describing a predetermined transient mode, e.g., by comprising the at least one transient parameter according to which the medical treatment is provided to the patient and/or the health state of the patient is monitored during the transient mode by the implant. The transient mode request may then comprise, e.g., only an identifier of a respective predetermined transient mode of the implant and the first and second time such as to instruct the implant to switch into that predetermined transient mode for the respective period. The implant may have only one predetermined transient mode or multiple predetermined transient modes which may, e.g., be adapted to different scenarios, health conditions, and/or patients.

For example, an implant may comprise a first set of parameters describing a predetermined transient mode in which the implant is switched off, a second set of parameters describing a predetermined transient mode in which the implant performs the same functions as in the standard operation mode but with adjusted parameters determining the operation in that transient mode, and/or a third set of parameters describing a predetermined transient mode in which at least one function the implant performs in the standard operation mode is deactivated (or activated). Any embodiment with further, adjusted, less and/or no predetermined transient modes is possible.

For example, the implant may further be configured to schedule operation in the transient mode between the first time and the second time upon receiving the transient mode request.

This increases the flexibility in when to provide the transient mode request such as, as long as it is provided before the first time, the attending health professional/implant specialist is not restricted in when to design and/or transmit the transient mode request to the implant. This may, e.g., occur hours, days, or weeks in advance.

In an exemplary embodiment of the implant, the means for receiving may further be configured to receive a rescheduling instruction having received the transient mode request and before the first time. The implant may be configured to, upon receiving the rescheduling instruction, cancel the scheduled transient mode and schedule a transient mode between a third time and a fourth time.

This may increase flexibility and the ability to react to, e.g., a re-scheduled surgery or any other shift of an associated event.

The respective rescheduling instruction may be a full new transient mode request with the instruction to cancel the scheduled transient mode to be rescheduled or a rescheduling instruction instructing the implant to reschedule the transient mode and adopt the transient parameters of the transient mode to be rescheduled.

The implant may be for example further configured to send a confirmation signal that the transient mode was scheduled and/or rescheduled.

The implant may for example send no confirmation but a notification when the transient mode could not be scheduled and/or rescheduled successfully. This may, e.g., occur when the implant specialist had accidentally entered a first or second time that lies in the past, a second time that is before the first time, etc. Then the attending implant specialist may retry or choose different settings, if, e.g., they had previously entered erroneous ones or parameters that lie without the possible range of the respective implant.

According to another aspect of the invention, an apparatus is provided that comprises means for sending a transient mode request to an implant. Therein, the transient mode request comprises one or more instructions instructing the implant to switch, a single time, into a transient mode from a first time until a second time and to switch back to the standard operation mode thereafter. Further, the transient mode may comprise at least one transient operation parameter different from at least one standard operation parameter of the standard mode.

With such apparatus, no time demanding in-office consultation is needed, e.g., for surgery preparation. The patient does not need to travel (potentially for hours) to the implant specialist, who does not need to spent time on such in-person consultation. Rather, the apparatus allows for remote control of the addressed implant receiving the transient mode request. Further, with this invention the timeframe where, e.g., therapy in the standard operation mode is deactivated can be limited, the implant can have therapy activated for longer.

An implant specialist may for example be informed about an upcoming surgery by the apparatus. Then, the implant specialist may create a new transient mode request with execution details for the implant. The apparatus may be, e.g., a remote server or an at least partly cloud-based server, and may transmit the transient mode request, optionally via a relay device and/or a patient device, to the implant. In the implant, the transient mode request is received and the schedule comprising the first time and second time for the surgery is set. Once the schedule is met, a special transient mode is activated (changed/deactivated therapy, changed/deactivated sensing, etc.). The implant may return to standard operation (by deactivating the transient mode and activating the standard operation mode) once the end of the schedule is met.

Generally, due to the close functional interaction between such apparatus and an implant as described herein, the functionalities described in reference to the implant may analogously apply to the apparatus and vice versa.

In an example, the transient mode request may indicate the first time and the second time for one-time use and/or may further comprise an instruction to use a predetermined mode of the implant as the transient mode.

Thereby, the apparatus may provide all relevant information to the implant receiving the transient mode request as required. This may be particularly relevant when, e.g., the first and/or second time are not identical with the start and end of the time slot associated with the upcoming event which the transient mode is being designed for. Executing only predetermined modes, on the other hand, may increase safety of the patient such as no unsuitable parameters may be requested (e.g., accidentally).

The apparatus may, e.g., receive the first and second time via user input of the attending implant specialist and/or determine the first and second time itself (semi-)automatically based on receiving the time slot of the upcoming event for which a suitable transient mode shall be scheduled.

For example, the apparatus may further comprise means for receiving a confirmation signal from the implant, the transient mode request is sent to, on whether the transient mode request was received and scheduled or not.

For the attending implant specialist or health professional operating the apparatus, this may be convenient feedback allowing them to assess whether their request was successful which may save time and increase the reliability of the respective process. In case of a fully automatic issuing of a transient mode request by the apparatus itself, this may apply analogously.

For example, the device may provide the as-received confirmation signal to the user via, e.g., a user interface of the apparatus, a web-interface, etc.

In an example, the apparatus may further comprise means for sending a rescheduling instruction after having sent the transient mode request and before the first time, wherein the rescheduling instruction comprises instructions to the implant to cancel the scheduled transient mode and to schedule a transient mode between a third time and a fourth time.

This prevents cases in which an already scheduled transient mode may not be required anymore but cannot be postponed or canceled.

Such rescheduling instruction may for example also comprise canceling the scheduled transient mode when, e.g., a planned surgery may be called off, without scheduling it at another (e.g., later) time.

In an example, the apparatus may further comprise an interface for receiving an instruction to send a transient mode request comprising at least a first time and a second time. Preferably, the apparatus may further be configured to, based on the instruction send, preferably automatically, the transient mode request and/or provide a suggestion and/or a call for a transient mode request via the interface.

For example, a call for a transient mode request may, e.g., be transmitted to an implant specialist to ask the implant specialist to set up and/or to provide a suitable transient mode request to be sent to the implant such as to be switched into a respective transient mode for an upcoming event.

In an example, two different interfaces may be used for receiving an instruction to send a transient mode request and for requesting and/or providing a suggestion and/or a call for a transient mode request.

For example, this interface may provide the possibility for the patient, e.g., via a patient app installed on a mobile phone, or an attending health professional for example scheduling a surgery of the patient, e.g., via an electronic health record (EHR) system, to input such call for a transient mode request which may then be transmitted to the respective implant specialist via the apparatus or only to the apparatus in case of an automated issuing of the transient mode request by the apparatus.

In an example, the apparatus may, in a semi-automatic embodiment, provide a suggestion for a transient mode request to the attending implant specialist based at least in part on the respective patient (and optionally their health record), implant type, timeslot of the respective event, etc. The attending implant specialist may then either accept the suggestion or modify it before transmitting it to the implant. Alternatively or additionally, the apparatus may output the call for a transient mode request, e.g., via a screen to the attending implant specialist, e.g., in form of a push notification or any alarm signal.

According to another aspect of the invention, a system is provided, wherein the system comprises an implant as described herein, and an apparatus as described herein.

Such system may yield various benefits described herein in reference to embodiments of the implant and/or the apparatus as a system comprising both counterparts provides synergistic benefits due to the mutual adjustment of the two counterparts working together in the system.

According to another aspect of the invention, a method comprising steps to be carried out by an implant is provided: The method may comprise operating in a standard mode, e.g. providing a medical treatment to a patient and/or monitoring a health state of the patient according to at least one standard operation parameter. It further comprises receiving a transient mode request. As a next step, it comprises, based thereon, switching, a single time, into a transient mode from a first time until a second time. In the transient mode, medical treatment may be provided to the patient and/or the health state of the patient may be monitored by the implant according to at least one transient parameter, wherein the at least one transient parameter is different from the at least one standard operation parameter. The final step is switching back to the standard operation mode thereafter.

This workflow bears all the benefits described herein in reference to the implant executing the respective steps and poses an efficient way to switch an implant into a transient mode for a limited time and then back into its standard operation mode.

According to another aspect of the invention, a method comprising steps carried out by an apparatus is provided. The method may comprise receiving a call for a transient mode request and sending a transient mode request to an implant. The transient mode request comprises one or more instructions instructing the implant to switch, a single time, into a transient mode from a first time until a second time and to switch back to the standard operation mode thereafter. Further, the transient mode may comprise at least one transient operation parameter different from at least one standard operation parameter of the standard mode.

This workflow bears all the benefits described herein in reference to the apparatus executing the respective steps and poses an efficient way to switch an implant into a transient mode for a limited time and then back into its standard operation mode.

Generally, any step of a method or functionality described in reference to the implant, the apparatus, and/or the respective system may be implemented as an instruction of a respective computer program to be executed by the respective implant, apparatus, and/or system.

Further, the various methods and functions outlined may be implemented in software, e.g. one or more computer programs comprising instructions which, when executed, carry out the steps of a method as described herein.

Further, the different methods described herein to be executed by the implant and/or the apparatus, respectively, may be combined at least in part to a combined method involving steps executed by the apparatus and the implant, respectively.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1A Schematic representation of an exemplary workflow informing an implant specialist.

FIG. 1B Schematic representation of an exemplary continued workflow of FIG. 1A for on-demand transient mode scheduling.

### DETAILED DESCRIPTION

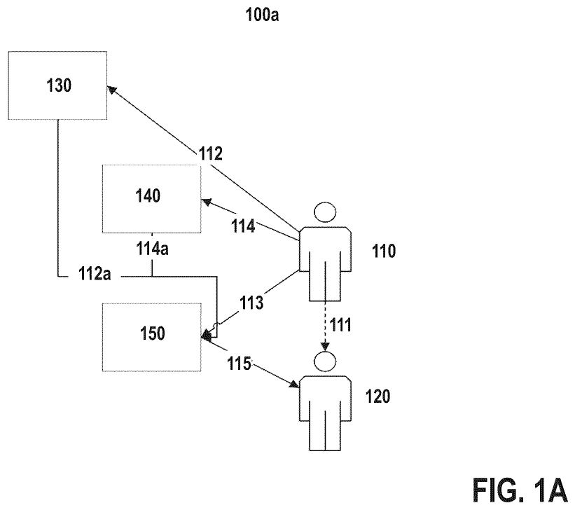

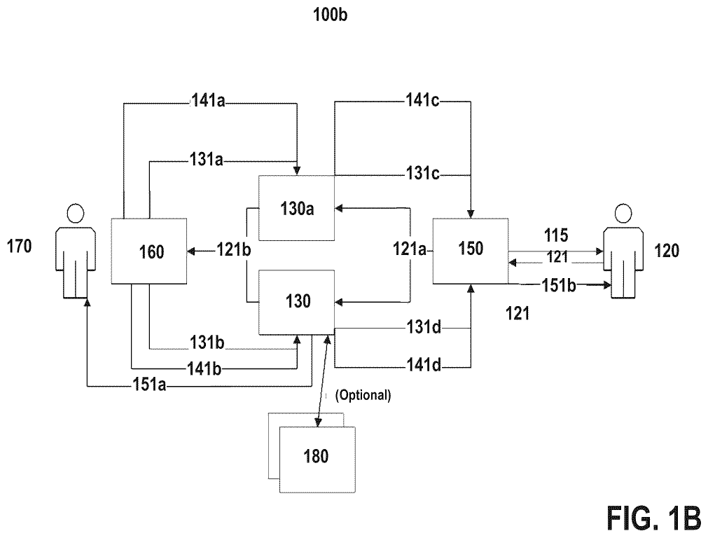

FIG. 1A shows a schematic representation of a workflow 100a of informing an implant specialist 120, e.g., a cardiologist, on an event requiring scheduling a transient mode for an implant.

In the exemplary embodiment of FIG. 1A, a surgery specialist 110 may inform the implant specialist 120 via either of the alternative information pathways 111, 112, 113, or 114. These pathways may be, e.g., direct or via a patient device running a patient application 130, an EHR 140, and/or a remote server 150.

Either way, the surgery specialist 110 may communicate all relevant surgery-related information. These may include the patient, the planned surgery, the time and duration of the surgery, surgery-specific details, comprising, e.g., planned tools and procedures, etc. to request the implant specialist 120 to assist in scheduling a surgery mode for one or more patient implants, e.g., a pacemaker. This may apply to any of the communication pathways described herein.

First, the surgery specialist 110 may directly inform 111 the implant specialist 120, e.g., during a phone call, by email, or in a personal meeting on the surgery details described herein. Optionally, this step may also be performed by the patient informing the implant specialist 120. As this does not involve the remote server 150, yet, the implant specialist 120 needs to enter the provided details in a special section of the remote server 150 where the surgery specialist 110 can enter details about the surgery like surgery specialist contact data, procedure, anesthesia needed, whether, e.g., magnetic resonance tomography, cardiac resynchronization therapy, and/or cauterization is planned/needed, time and duration of, e.g., a surgery, etc., when the communicated information shall be entered into the remote server, e.g., to be stored in an electronic patient file for example of the HER 140 and/or at the remote server 150. The remote server 150 may, e.g., comprise a user interface or a mask to input the relevant data.

Second, the surgery specialist 110 may provide a data packet 112 comprising information on the planned surgery as described herein to a patient application 130, e.g., remotely via wireless communication or directly via, e.g., a remote device like a mobile phone or a computer running the patient application 130. The patient application 130 may provide a special input section where the patient and/or the surgery specialist 110 can enter details about the surgery. The patient application 130 may then provide the as-received data 112 to the remote server 150 as a data packet 112a, which may be identical to the data packet 112 or a processed version thereof, e.g., it may comprise more information or less information than comprised in the data packet 112.

Third, the surgery specialist 110 may provide direct input 113 into the remote server, e.g., triggered by the patient application 130. For this, the patient application 130 may, e.g., provide a one-time usable special code (for example a QR code or a barcode) that can be scanned by the surgery specialist 110 and forwards to a special section of the remote server 150 where the surgery specialist 110 can enter details about the surgery as described herein. The remote server 150 may, e.g., comprise a user interface or a mask to input the relevant data, as described above.

Last, the surgery specialist 110 may provide a data packet 114 comprising details about the surgery as described herein into the EHR 140 which may forward an identical data packet 114a or a processed version 114a of the data packet 114 to the apparatus 150, which in the example of FIG. 1A is a remote server 150.

The remote server 150 may thus receive any of the data packets 112a, 113, and/or 114a and transmit a further data packet 115 comprising the identical information, combined versions, processed versions, and/or parts thereof to the implant specialist 120, e.g., via their computer connected to the remote server 150. Data packets 112a, 113, and/or 114a may comprise an instruction to send a transient mode request. The instruction may comprise the first and second time to be used in the transient mode request. In other examples, it may comprise times of an event, e.g. of a planned surgery, such that the first and second time for the transient mode request may be determined based thereon, and optionally based on additional information, e.g. contained in the instruction and/or the EHR of the patient etc. (e.g. about the event and/or the patient).

The data packet 115 may act as a call for a transient mode request to be issued, entered, etc. by the implant specialist. Such exemplary call for a transient mode request may, e.g., ask the implant specialist to set-up and/or provide a suitable transient mode request. In some examples, additionally or alternatively, a suggestion for a transient mode request may be provided, e.g. to the implant specialist.

In an exemplary (semi)automatic system, e.g., the remote server 150 itself may (semi)automatically provide such suitable transient mode request in response to an according instruction to send a transient mode request. In other examples, the remote server 150 may wait for a transient mode request to be received, e.g. from the implant specialist.

In all cases above, the remote server may add the received information to a patient file in the EHR 140 and an alarm may be raised in the system. The attending implant specialist may receive the alarm/request and can see all related data for the surgery.

This poses the initiating step for a workflow 100a continued in FIG. 1B.

FIG. 1B shows a schematic representation of a workflow 100b that may continue the workflow of FIG. 1A for on-demand transient mode scheduling of an implant 160 of a patient 170. Generally, the communication may involve a patient device 130, e.g., running a patient application, and/or a relay device 130a. The following relates only to the patient device 130 but may analogously also apply to embodiments comprising a relay device 130a in addition or alternatively to the patient device 130.

FIG. 1B shows the last step of FIG. 1A, wherein the implant specialist 120 receives data 115 from the remote server 150 comprising information on the surgery as described herein.

Based thereon, the implant specialist 120 may design a suitable transient mode request 121 and provide it to the remote server 150. In other embodiments, this may occur fully automatically by the remote server 150 itself (e.g. based on the data 150). Optionally, the implant specialist 120 may be enabled to confirm seeing the request in the remote server 150. The confirmation is forwarded to the surgery specialist 110 who may stay involved in the process. In another example, the transient mode request 121 may only comprise information on time and duration and the implant 160 itself may already comprise all relevant instructions for the transient mode (this may, e.g., have been set during manufacturing) and the implant specialist 120 may then only activate it. Additionally, the predetermined transient mode may be customized, e.g., by adding parameters to the transient mode request 121 such as, for example instructions on whether an ICD may shock or not, whether backup stimulation may be enabled or not, etc.

It is noted that the data 150 is optional. In other examples, a transient mode request may be entered into remote server 150 (or a corresponding interface to it), e.g. by an implant specialist, as a first step. This transient mode request may be based on knowledge, by the implant specialist, on an upcoming event which they may have obtained by other sources.

The transient mode request 121a is then, independent of the content of the transient mode request 121, transmitted from the remote server 150 to the patient device 130 and/or the relay device 130a. The transient mode request 121a may be an identical or a modified version of the transient mode request 121 received by the remote server 150. The patient device 130 and/or the relay device may then transmit the transient mode request 121b to the implant 160. The transient mode request 121b may be an identical or a modified version of the transient mode requests 121 and 121a. Independent on whether the transient mode request 121 is forwarded directly or altered by the remote server 150, the patient device 130 and/or the relay device 130a, the transient mode request 121 may be end-to-end encrypted between remote server 150 and implant 160, e.g., such that the patient device 130 cannot read the content. The implant 160 may receive the transient mode request 121b and may be able to set a schedule for replacing the standard operation mode with the transient operation mode. The transient mode may comprise, e.g., diagnostic-therapeutic settings, statistic and recording parameters to special surgery mode (for example every sensed event is tagged as “during surgery”, electrocardiography recording can be extended or restricted, etc.). Alternatively, it may disable the whole therapy or parts of the therapy (e.g., shock output for implantable cardioverter-defibrillators (ICDs), safe ventricular (VVI) backup stimulation, switch the implant into a magnetic resonance imaging-compatible mode, etc.) that would disturb the surgery.

Optionally, the transient mode request 121 may also be transmitted directly (not shown) to the patient device 130. The patient device 130 may receive it and set a schedule on its own. Once the schedule is met, the patient device 130 may notify the patient 170. This optional step would give more control to the patient 170. In case of, e.g., any schedule deviations, the patient 170 would be reminded about the procedure and could intervene.

The implant 160 then sends a response on whether set up of the transient mode according to the transient mode request 121b was successful or not. This may occur, e.g., immediately or within 5 or 10 minutes and comprise sending the response 131a, 131b back to the patient device 130 and/or the relay device 130a. From there, the response or a modified version thereof 131c, 131d may be transmitted to the remote server 150. Alternatively, the response 131a, 131b may be transmitted directly from the implant 160 to the remote server 150. The response may, e.g., be seen by the patient 170 via the patient device 130 and/or by the implant specialist 120 via the remote server 150. Optionally, the result of the transient mode request 121 (e.g., comprising details of the newly set and scheduled transient mode) is also forwarded to the surgery specialist who may thus stay involved in the process. The responses 131a, 131b, 131c, 131d may be confirmation signals 131 to confirm that the transient mode was scheduled and/or rescheduled.

When the response 131a, 131b, 131c, 131d is positive indicating that the transient mode was scheduled successfully, no further action by the patient 170 or the implant specialist 120 is required. Should it be negative, the implant specialist 120 may re-try by sending another transient mode request 121. The implant may then switch into the transient mode, e.g., during a planned surgery, and switch back into the standard operation mode after the surgery as scheduled automatically and without the need of intervention be the patient 170, the surgery specialist 110, and/or the implant specialist 120.

Once the schedule start is reached (in case of an exemplary surgery, usually hours before the surgery is performed), the implant 160 may automatically start the transient mode and ends it automatically according to the schedule end (in case of an exemplary surgery, usually hours after the surgery is performed). The implant 160 may then send the results 141a, 141b of both actions to the patient device 130 where the patient may see it and/or the relay device 130a. They may transmit identical and/or modified versions thereof 141c, 141d to the remote server 150 where the implant specialist 120 and the surgery specialist 110 may see it.

Optionally, the patient device 130 may collect data, e.g., physiological patient data, for example from any other connected (wearable) device 180 (for example a smart watch) specifically for the transient mode timeframe and can transmit the data to the remote server.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implant configured to:
provide a medical treatment to a patient and/or monitor a health state of the patient according to at least one standard operation parameter, when in a standard operation mode;
the implant comprising means for receiving a transient mode request;
wherein receiving the transient mode request causes the implant to switch, a single time, into a transient mode from a first time until a second time and to switch back to the standard operation mode thereafter.

2. The implant of claim 1, wherein the transient mode request indicates the first time and the second time for one-time use.

3. The implant of claim 1, wherein the transient mode request further comprises an instruction to use a predetermined mode of the implant as the transient mode.

4. The implant of claim 1, further configured to schedule operation in the transient mode between the first time and the second time upon receiving the transient mode request.

5. The implant of claim 4, wherein the means for receiving are further configured to receive a rescheduling instruction after having received the transient mode request and before the first time, wherein the implant is configured to, upon receiving the rescheduling instruction, cancel the scheduled transient mode and schedule a transient mode between a third time and a fourth time.

6. The implant of claim 4, further configured to send a confirmation signal that the transient mode was scheduled and/or rescheduled.

7. An apparatus, comprising:
means for sending a transient mode request to an implant;
wherein the transient mode request comprises one or more instructions instructing the implant to switch, a single time, into a transient mode from a first time until a second time and to switch back to a standard operation mode thereafter;
wherein the transient mode preferably comprises at least one transient operation parameter different from at least one standard operation parameter of the standard operation mode.

8. The apparatus of claim 7, wherein the transient mode request indicates the first time and the second time for one-time use; and/or wherein the transient mode request further comprises an instruction to use a predetermined mode of the implant as the transient mode.

9. The apparatus of claim 7, further comprising means for receiving a confirmation signal, from the implant the transient mode request is sent to, on whether the transient mode request was received and scheduled or not.

10. The apparatus of claim 7, further comprising means for sending a rescheduling instruction after having sent the transient mode request and before the first time, wherein the rescheduling instruction comprises instructions to the implant to cancel the scheduled transient mode and to schedule a transient mode between a third time and a fourth time.

11. The apparatus of claim 7, further comprising an interface for receiving an instruction to send a transient mode request; the apparatus preferably further configured to, based on the instruction, send, automatically, the transient mode request and/or provide a suggestion and/or a call for a transient mode request via the interface.

12. A system comprising an implant according to claim 1 and an apparatus comprising:
means for sending a transient mode request to an implant;
wherein the transient mode request comprises one or more instructions instructing the implant to switch, a single time, into a transient mode from a first time until a second time and to switch back to a standard operation mode thereafter;
wherein the transient mode preferably comprises at least one transient operation parameter different from at least one standard operation parameter of the standard operation mode.

13. A method comprising the following steps carried out by an implant:
receiving a transient mode request;
based thereon, switch, a single time, into a transient mode from a first time until a second time; and
switching back to a standard operation mode thereafter.

14. A method comprising the following steps carried out by an apparatus sending a transient mode request to an implant; wherein the transient mode request comprises one or more instructions instructing the implant to switch, a single time, into a transient mode from a first time until a second time and to switch back to a standard operation mode thereafter.
